(19) United States
(12) Patent Application Publication
(10) Pub. No.: US 2002/0022652 A1
(43) Pub. Date: Feb. 21, 2002

(54) Methylnogarol compositions and uses thereof

(76) Inventor: Dennis M. Brown, Menlo Park, CA (US)

Correspondence Address: FLEHR HOHBACH TEST ALBRITTON & HERBERT LLP, Four Embarcadero Center - Suite 3400, San Francisco, CA, 94111-4187, US

(21) Appl. No.: 09/834,180

(22) Filed: Apr. 12, 2001

Related U.S. Application Data
(60) Provisional application No. 60/197,106, filed on Apr. 12, 2000.

(51) Int. Cl. A61K 33/24; A61K 31/357; A61K 31/337

(52) U.S. Cl. 514/452; 514/449; 514/283; 424/649

(57) ABSTRACT

A method of treatment of a host with a cellular proliferative disease, comprising contacting the host with a methylnogarol and an antiproliferative agent, each in an amount sufficient to modulate said cellular proliferative disease, is described. In some embodiments the methylnogarol comprises menogaril (7-O-Methylnogarol; 7-Con-O-Methylnogarol; U-52047; 2,6-Epoxy-2H-naphthaceno[1,2-b]oxocin-9,16-dione, 4-(dimethylamino)-3,4,5,6,11,12,13,14-octahydro-3,5,8,10,13-pentahydroxy-11-methoxy-6,13 -dimethyl, -(2alpha,3beta,4alpha,5beta,6alpha, 11 alpha, 13alpha)-, (P)-; 2,6-Epoxy-2H-naphthaceno[1,2-b]oxocin-9,16-dione,4-(dimethylamino)-3,4,5,6,11,12,13,14-octahydro-3,5,8,10,13-pentahydroxy-11-methoxy-6,13-dimethyl-; 7(R)-O-Methylnogarol; 7-OMEN). Antiproliferative agents of the invention comprise alkylating agents, intercalating agents, metal coordination complexes, pyrimidine nucleosides, purine nucleosides, inhibitors of nucleic acid associated enzymes and proteins, and agents affecting structural proteins and cytoplasmic enzymes. The invention comprises the described methods as well as compositions comprising a methylnogarol and an antiproliferative agent.

This application claims the benefit of U.S. Provisional Application No. 60/197,106, filed Apr. 12, 2000.

### FIELD OF THE INVENTION

The technical field of the invention is the use of methylnogarols with antiproliferative agents to treat a host with a cellular proliferative disease.

### BACKGROUND OF THE INVENTION

There is considerable interest in modulating the efficacy of currently used antiproliferative agents to increase the rates and duration of antitumor effects associated with conventional antineoplastic agents.

Conventional antiproliferative agents used in the treatment of cancer are broadly grouped as chemical compounds which (1) affect the integrity of nucleic acid polymers by binding, alkylating, inducing strand breaks, intercalating between base pairs or affecting enzymes which maintain the integrity and function of DNA and RNA; (2) chemical agents that bind to proteins to inhibit enzymatic action (e.g. antimetabolites) or the function of structural proteins necessary for cellular integrity (e.g. antitubulin agents). Other chemical compounds that have been identified to be useful in the treatment of some cancers include drugs which block steroid hormone action for the treatment of breast and prostate cancer, photochemically activated agents, radiation sensitizers and protectors.

Of special interest to this invention are those compounds that directly affect the integrity of the genetic structure of the cancer cells. Nucleic acid polymers such as DNA and RNA are prime targets for anticancer drugs. Alkylating agents such as nitrogen mustards, nitrosoureas, aziridine containing compounds directly attack DNA. Metal coordination compounds such as cisplatin and carboplatin similarly directly attack the nucleic acid structure resulting in lesions that are difficult for the cells to repair which, in turn, can result in cell death. Other nucleic acid affecting compounds include anthracycline molecules such as doxorubicin, which intercalates between the nucleic acid base pairs of DNA polymers, bleomycin which causes nucleic acid strand breaks, fraudulent nucleosides such as pyrimidine and purine nucleoside analogs which are inappropriately incorporated into nucleic polymer structures and ultimately cause premature DNA chain termination. Certain enzymes that affect the integrity and functionality of the genome can also be inhibited in cancer cells by specific chemical agents and result in cancer cell death. These include enzymes that affect ribonucleotide reductase (e.g., hydroxyurea, gemcitabine), topoisomerase I (e.g., camptothecin) and topoisomerase II (e.g., etoposide).

One of the most broadly used of these DNA targeted anticancer drugs is cisplatin (cis-diamminedichloroplatinum II). This compound is active against several human cancers including testicular, small-cell lung, bladder, cervical and head and neck cancer. In addition, other drugs, such as the camptothecins which affect topoisomerase I, and paclitaxel, which affects tubulin protein in cells, are used in the treatment of human cancers.

While the clinical activity of currently approved antiproliferative agents against these forms of cancers are demonstratable, improvements in tumor response rates, duration of response and ultimately patient survival are still sought. The invention described herein demonstrates the novel use of methylnogarols and derivatives including menogaril which can potentiate the antitumor effects of chemotherapeutic drugs, in particular, agents affecting the integrity of nucleic polymers such as DNA.

### SUMMARY OF THE INVENTION

Methods and compositions are provided for the treatment of a host with a cellular proliferative disease, particularly a neoplasia. In the subject methods, a pharmaceutically acceptable methylnogarol and an antiproliferative agent are administered in an amount sufficient to modulate the cellular proliferative disease.

### DETAILED DESCRIPTION OF THE FIGURES

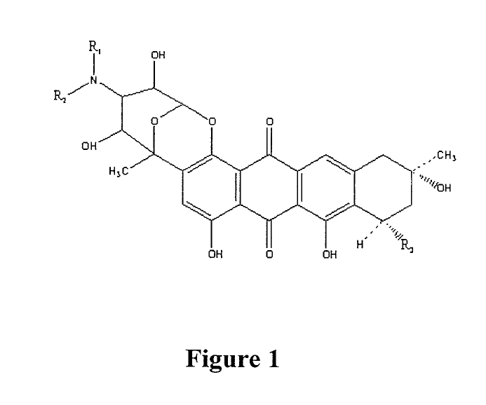

FIG. 1 depicts the general structure of a methylnogarol analog. R₁ through R₃ represent possible substitution groups.

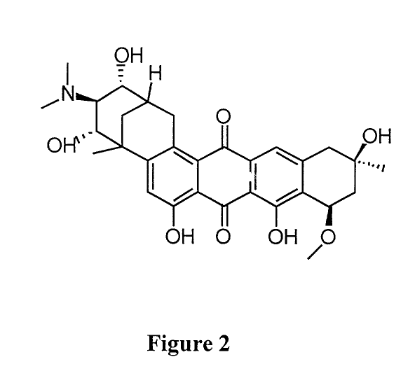

FIG. 2 shows the structure of the methylnogarol analog,

menogaril.

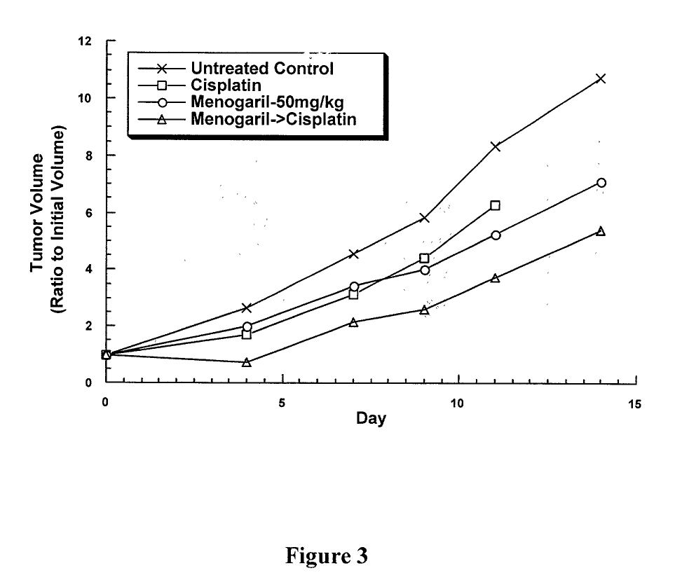

FIG. 3 shows tumor growth delay, as tumor volume on days after treatment with menogaril, menogaril followed by cisplatin, or cisplatin alone.

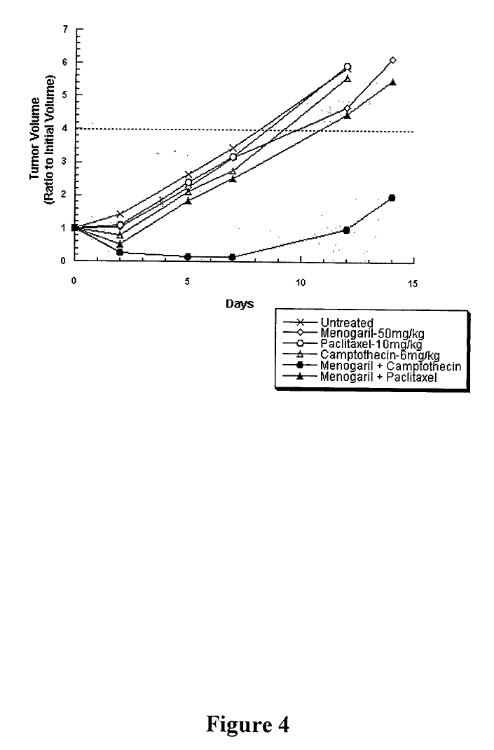

FIG. 4 shows tumor growth delay, as tumor volume on days after treatment with menogaril, paclitaxel, camptothecin, menogaril followed by camptothecin, or menogaril followed by paclitaxel.

### DETAILED DESCRIPTION OF THE INVENTION

Methods and compositions are provided for the treatment of a host with a cellular 5 proliferative disease, particularly a neoplasia. In the subject methods, a pharmaceutically acceptable methylnogarol is administered, preferably systemically, in conjunction with an antiproliferative agent to improve the anticancer effects. In a preferred embodiment, the methylnogarol provides a chemopotentiator effect.

The agents are provided in amounts sufficient to modulate a cellular proliferative disease. In one embodiment, modulation of a cellular proliferative disease comprises a reduction in tumor growth. In another embodiment, modulation of a disease comprises inhibition of tumor growth. In another embodiment, modulation of a cellular proliferative disease comprises an increase in tumor volume quadrupling time (described below). In another embodiment, modulation of a cellular proliferative disease comprises a chemopotentiator effect. In another embodiment, modulation of a disease comprises a chemosensitizing effect. In other embodiments, modulation of a disease comprises cytostasis. In still other embodiments, modulation of a disease comprises a cytotoxic effect.

A chemical agent is a “chemopotentiator” when it enhances the effect of a known antiproliferative drug in a more than additive fashion relative to the activity of the chemopotentiator or antiproliferative agent used alone. In some cases, a chemosensitizing effect may be observed. This is defined as the effect of use of an agent that if used alone would not demonstrate significant antitumor effects but would improve the antitumor effects of an antiproliferative agent as compared to the antiproliferative agent by itself.

As used herein, the term “methylnogarol” includes all members of that chemical family including the desmethyl forms and analogs thereof. The methylnogarol family is defined by chemical structure as the ring structures in FIG. 1.

A methylnogarol analog is further defined but not limited to the structure depicted in FIG. 1, having substituent or substitute groups at R₁ through R₃. Table 1 lists some possible structures of R₁ through R₃ for methylnogarol analogs. R group substitutions are typically employed to improve biological activity, pharmaceutical attributes such as bioavailability or stability, or decrease toxicity. In one embodiment, R groups include alkyl substitutions (e.g., methyl, ethyl, propyl etc.). In another embodiment, R groups include alkoxy (e.g., methoxy, ethoxy, butoxy, etc.). Substitutions at R₁ through R₃ are not limited to the above examples, however.

TABLE 1

| Group | Substitution | Length |
|---|---|---|
| R₁ | Alkyl | C₁→C₅ |
| R₂ | Alkyl | C₁→C₅ |
| R₃ | Alkoxy | OC₁→OC₅ |
| | Hydrogen | |

A methylnogarol analog is a further chemical refinement. A specific example of methylnogarol is menogaril which is also known by the following chemical synonyms: 7-0-Methylnogarol; 7-Con-O-Methylnogarol; U-52047; 2,6-Epoxy-2H-naphthaceno[1,2-b]oxocin-9,16-dione, 4-(dimethylamino)-3,4,5,6,11,12,13,14-octahydro-3,5,8,10,13-pentahydroxy-11-methoxy-6,13-dimethyl-,(2alpha,3beta,4alpha,5beta,6alpha, 11 alpha, 13alpha)-, (P)-; 2,6-Epoxy-2H-naphthaceno[1 ,2-b]oxocin-9, 16-dione,4-(dimethylamino)-3,4,5,6,11,12,13,1 4-octahydro-3,5,8,10,13-pentahydroxy-11-methoxy-6,13-dimethyl-; 7(R)-O-Methylnogarol; 7-OMEN (FIG. 1). Menogaril has the structure shown in FIG. 2.

As used herein, antiproliferative agents are compounds which induce cytostasis or cytotoxicity. “Cytostasis” is the inhibition of cells from growing, while “cytotoxicity” is defined as the killing of cells. Specific examples of antiproliferative agents include: antimetabolites, such as methotrexate, 5-fluorouracil, gemcitabine, cytarabine, pentostatin, 6-mercaptopurine, 6-thioguanine, L-asparaginase, hydroxyurea, N-phosphonoacetyl-L-aspartate (PALA), fludarabine, 2-chlorodeoxyadenosine, and floxuridine; structural protein agents, such as the vinca alkaloids, including vinblastine, vincristine, vindesine, vinorelbine, paclitaxel, and colchicine; antibiotics, such as dactinomycin, daunorubicin, doxorubicin, idarubicin, bleomycins, plicamycin, and mitomycin; hormone antagonists, such as tamoxifen and luteinizing hormone releasing hormone (LHRH) analogs; nucleic acid damaging agents such as the alkylating agents mechlorethamine, cyclophosphamide, ifosfamide, chlorambucil, dacarbazine, methylnitrosourea, semustine (methyl-CCNU), chlorozotocin, busulfan, procarbazine, melphalan, carmustine (BCNU), lomustine (CCNU), and thiotepa, the intercalating agents doxorubicin, dactinomycin, daurorubicin and mitoxantrone, the topoisomerase inhibitors etoposide, camptothecin and teniposide, agents that affect tubulin, such as paclitaxel, and the metal coordination complexes cisplatin and carboplatin.

The following examples are offered by way of illustration and not by way of limitation.

### EXAMPLES

### Example 1

### The chemopotentiation of cisplatin (CDDP) by menogaril

Transplantable experimental murine fibrosarcomas (2×10⁵ RIF−1 cells) were grown intradermally in the flanks of 3 month old female C3H mice (Charles River, Holister, Calif.). When the tumors reached a volume of approximately 100 mm³, the mice were randomly assigned to each experimental group (4 mice per group).

The experimental compositions were prepared as described in Table 2.

TABLE 2

| Agent | Dose | Solvent | Supplier |
|---|---|---|---|
| Menogaril | 50 mg/kg | DMSO | NCI |
| Cisplatin | 4 mg/kg | Water for Injection | David Bull Labs |

The chemopotentiator, menogaril, was obtained from NCI and was made to the appropriate concentration in DMSO. Cisplatin (David Bull Laboratories- Mulgrave, Australia, lot. 5201844x) was made to the appropriate concentration in water for injection. The compositions were injected systemically (i.e., intraperitoneally, i.p.), in a volume of 100 microliters. For the treatment of group 3, the chemopotentiator, menogaril, was injected 30 minutes prior to the injection of cisplatin. After treatment, the growth of the tumors was monitored three times per week by caliper measurements of three perpendicular diameters of the tumor and calculation of tumor volume from the formula:

V=π/6×D₁×D₂×D₃,

were D₁₋₃ is in mm.

The tumors were followed until they reached a size of four times their day zero treatment volume (TVQT), or up to 30 days after treatment, whichever came first. The data is expressed as the “tumor volume quadrupling time” (TVQT) mean and as the “delay.” Mean TVQT is the mean days required for individual tumors to grow to four times the tumor volume at the initial treatment day. The “delay” is the median of days required for a tumor to grow to four times the mean size of the treated group, minus the median of days required to grow to four times the mean size of the control group. The data is also expressed as the ratio of the tumor volume quadrupling time of the treated tumor over the untreated control group (TVQT/CTVQT). Increasing values of this ratio indicate increased antitumor response.

The data is presented in Table 3 below and in FIG. 3.

TABLE 3

| | Treat- | Dose | Mean TVQT ± | TVQT/ | Median | Delay |
|---|---|---|---|---|---|---|
| Group | ment | (mg/kg) | S.E. | CTVQT | (TVQT) | (Days) |
| 1 | Un- | — | 6.3 ± 0.3 | 1.0 | 6 | 0.00 |
| | treated | | | | | |
| | Control | | | | | |
| 2 | Meno- | 50 | 9.1 ± 0.5 | 1.4 | 9.4 | 3.31 |
| | garil | | | | | |
| 3 | Meno- | 50 → 4 | 11.7 | 1.9 | 11.8 | 5.78 |
| | garil → | | | | | |
| | Cispla- | | | | | |
| | tin | | | | | |
| 4 | Cispla- | 4 | 8.4 ± 0.3 | 1.3 | 8.1 | 2.10 |
| | tin | | | | | |

The arrow (→) in Group 3 indicates administration 30 minutes following administration of menogaril.

The results of Table 3 indicate that the antiproliferative activity of cisplatin is enhanced by the use of the chemopotentiator, menogaril in that a more than additive effect was observed when both compounds were used to treat the tumor bearing mice (group 3) in comparison to the use of cisplatin alone (group 4) or menogaril alone (group 2).

### Example 2

### Enhancement of camptothecin and paclitaxel by menogaril

The ability of menogaril to enhance the effect of camptothecin or paclitaxel was also studied, using the same methods described in Example 1. Table 4 illustrates the results of experiments for 6 groups of animals. For each injection, 100 microliters was injected. In Group 5, two animals died at day 5 and 1 died at day 8. For group 6, 1 animal died at day 5. FIG. 4 shows the results as tumor volume on days after treatment with menogaril, paclitaxel, camptothecin, menogaril followed by camptothecin, or menogaril followed by paclitaxel.

TABLE 4
Menogaril Enhancement of Camptothecin and Paclitaxel

| | | Number | Route of | | Mean | | | |
|---|---|---|---|---|---|---|---|---|
| | | of | Admini- | Dose | TVQT (± | TVQT/ | Median | |
| Group | Treatment | Tumors | stration | (mg/kg) | SE) | CTVQT | (TVQT) | Delay |
| 1 | Untreated | 8 | — | — | 8.7 ± 0.5 | 1.0 | 8.4 | 0.00 |
| 2 | Menogaril | 8 | IP | 50 | 10.2 ± 0.8 | 1.2 | 10.2 | 1.84 |
| 3 | Paclitaxel | 8 | IP | 10 | 9.0 ± 0.4 | 1.0 | 8.7 | 0.36 |
| 4 | Camptothecin | 8 | IP | 6 | 9.9 ± 0.5 | 1.1 | 9.8 | 1.44 |
| 5 | Menogaril → | 2/8 | IP | 50 → 6 | 17.4 ± 0.2 | 2.0 | 17.4 | 9.05 |
| | Camptothecin | | | | | | | |
| 6 | Menogaril → | 6/8 | IP | 50 → 10 | 11.5 ± 0.8 | 1.3 | 12 | 3.60 |
| | Paclitaxel | | | | | | | |

The arrow (→) in Groups 5 and 6 indicates administration 30 minutes following administration of menogaril.

We claim:

1. A method of treatment of a host with a cellular proliferative disease, comprising contacting said host with a methylnogarol and an antiproliferative agent, each in an amount sufficient to modulate said cellular proliferative disease.

2. The method according to claim 1, wherein said methylnogarol comprises menogaril.

3. The method according to claim 1, wherein said methylnogarol comprises a menogaril analog.

4. The method according to claim 1 wherein said antiproliferative agent comprises an agent that interacts with nucleic acids or an agent that interacts with proteins.

5. The method according to claim 1 wherein said antiproliferative agent comprises an alkylating agent, an intercalating agent, a metal coordination complex, a pyrimidine nucleoside, a purine nucleoside, an inhibitor of nucleic acid associated enzymes, or an inhibitor of nucleic acid associated proteins.

6. The method according to claim 1 wherein said antiproliferative comprises cisplatin, paclitaxel, or camptothecin.

7. A method according to claim 1 when said methylnogarol is administered before the administration of said antiproliferative agent.

8. A method according to claim 1 when said methylnogarol is administered during the administration of said antiproliferative agent.

9. A method according to claim 1 when said methylnogarol is administered after the administration of said antiproliferative agent.

10. The method of claim 1 wherein the effect on said disease with said composition is greater than that for said antiproliferative agent alone.

11. A composition comprising a methylnogarol and an antiproliferative agent.

12. The composition of claim 11 wherein said methylnogarol comprises menogaril.

13. The composition of claim 11 wherein said antiproliferative agent comprises cisplatin, paclitaxel or camptothecin.

14. Use of a methylnogarol and an antiproliferative agent in the formulation of a medicament for the treatment of a cellular proliferative disease.
